(19) United States
(12) Patent Application Publication
(10) Pub. No.: US 2016/0135751 A1
(43) Pub. Date: May 19, 2016

(54) SYSTEM AND METHOD FOR DETECTING NEUROMOTOR DISORDER

(71) Applicant: ARIZONA BOARD OF REGENTS FOR THE UNIVERSITY OF ARIZONA, Tucson, AZ (US)

(72) Inventors: Joseph S. Valacich, Tucson, AZ (US); Jeffrey L. Jenkins, Provo, UT (US); John Howie, Seattle, WA (US)

(73) Assignee: Arizona Board of Regents for the University of Arizona, Tucson, AZ (US)

(21) Appl. No.: 14/900,163

(22) PCT Filed: Jun. 20, 2014
(86) PCT No.: PCT/US14/43527

§ 371 Date: Dec. 20, 2015

Related U.S. Application Data
(60) Provisional application No. 61/838,143, filed on Jun. 21, 2013.

(51) Int. Cl. A61B 5/00; A61B 5/11

(52) U.S. Cl. CPC A61B 5/6897; A61B 5/11; A61B 5/4082; A61B 5/4088; A61B 5/4227; A61B 5/4244; A61B 5/746; A61B 5/747; A61B 5/6898

(57) ABSTRACT

The present invention provides a system and a method for detecting a neuromotor disorder or condition in a subject using an electronic device that comprises an input device. The method generally involves determining input device usage characteristics of the subject and comparing the result to a reference input device usage characteristic.

### CROSS-REFERENCE TO RELATED APPLICATIONS

This application claims the priority benefit of U.S. Provisional Application No. 61/838,143, filed Jun. 21, 2013, which is incorporated herein by reference in its entirety.

### FIELD OF THE INVENTION

The present invention relates to system and a method for detecting a neuromotor disorder in a subject using an electronic device that comprises an input device. In particular, the method is based on motion of the subject's hand and/or finger. The method generally involves determining input device usage characteristics of the subject and comparing the result to a reference input device usage characteristic.

### BACKGROUND OF THE INVENTION

Hand tremors are an early symptom of many possible medical or clinical conditions, e.g., hypoglycemia, neurological disorders, reactions to mediations, alcohol abuse or withdrawal, mercury poisoning, overactive thyroid, clinical trial side effects, peripheral neuropathy, early Parkinson's Disease, early Alzheimer's Disease, and liver failure. Such conditions are referred to herein simply as a neuromotor disorder. However, it should be appreciated that the term neuromotor disorder refers to any clinical disorder that results in changes to one's fine motor skill.

Early detection of some neuromotor disorders, e.g., those caused by diabetes, can be extremely beneficial to the subject as it allows the subject to take an appropriate action prior to onset of a more serious clinical condition. While many methods are available for determining a neuromotor disorder in a subject, conventional methods typically require active monitoring, e.g., determining glucose level by checking one's blood sugar level, or an actual clinical test that requires visit to a doctor's office. Such methods generally take time away from one's normal routine or task and can be time consuming as well as being costly.

Therefore, there is a need for an unobtrusive method of determining a neuromotor disorder in a subject.

### SUMMARY OF THE INVENTION

Some aspects of the invention provide unobtrusive method for determining neuromotor disorder in a subject by monitoring the subject's input device usage characteristic and comparing the result with a reference input device usage characteristic. The reference input device usage characteristic is generally the subject's own input device usage characteristic that was determined within a year, typically a month, more typically within a week, often within the 24 hour period or most often within an hour. By detecting changes in the input device usage characteristics, one can determine whether the subject has a neuromotor disorder.

In some instances, the reference input device characteristic is the input device usage characteristic of the subject during the same or substantially the same (i.e., within an hour) time of the day. For example, if the current input device usage characteristic is determined at 9 o'clock in the morning, the reference input device usage characteristic is that taken from about 8 o'clock to about 10 o'clock in the morning the previous day. In this manner, substantially the same time period of the day (i.e., ±2 hour, often ±1 hour) results are compared. In some instances, the reference input device usage characteristic is the input device usage characteristic of the subject determined same day of the week. For example, the input device usage characteristic of the subject on Monday morning is compared to the subject's input device usage characteristic that was determined Monday morning of the previous week.

One aspect of the invention provides a monitoring system for detecting or determining a change in a clinical condition of a subject while using a central processing unit based electronic device comprising an input device that is capable of detecting motion of a subject's hand or finger. The system includes: a data interception unit configured to intercept inputs from a user, wherein the data interception unit is configured to passively collect an input device usage characteristics; a behavior analysis unit operatively connected to said data interception unit to receive the passively collected input device usage characteristic, and a behavior comparison unit operatively connected to said behavior analysis unit, wherein said monitoring system dynamically monitors and passively collects input device usage characteristic information, and translates said input device usage characteristic information into representative data, stores and compares different results (or to a reference input device usage characteristic), and outputs a result associated with changes in the clinical condition of the subject.

In some embodiments, the clinical condition comprises hypoglycemia, a neurological disorder, a reaction to mediation, alcohol abuse or withdrawal, mercury poisoning, overactive thyroid, a clinical trial side-effect, peripheral neuropathy, early Parkinson's Disease, early Alzheimer's Disease, liver failure, or other conditions that will cause a change in hand movements. In one particular embodiment, the clinical condition is hypoglycemia.

Yet in other embodiments, the input device is a pointing device. In some instances, the pointing device comprises mouse, touch screen, track ball, touch pad, joystick, stylus, or a combination thereof. In some cases, said pointing device usage characteristic comprises: number of changes in direction on the x axis (x-flips); number of changes in direction on the y axis (y-flips); changes in speed; changes in precision; changes in distance; changes in idle time; changes in reaction time; changes in response time; changes in area under the curve; changes in maximum deviation; changes in additional distance; changes in normalized jerk; changes in keystroke transition time; changes in keystroke dwell time; changes in acceleration; changes in click latency; or a combination thereof.

Still in other embodiments, said behavior comparison unit is further configured to alert the subject when the result indicates hypoglycemic condition or other condition that influences fine motor control, such as hand movements. Exemplary alerts include a pop-up window, text message to the subject, an automated phone call, a phone call from health care provider, email, and a combination thereof.

In other embodiments, said monitoring system is configured in a computer, a smartphone, or any other central processing unit based electronic device comprising an input device that is capable of detecting a motion input from the subject's hand or finger.

Still yet in other embodiments, said monitoring system is suitably configured for real-time monitoring.

Another aspect of the invention provides a method of determining change in a clinical condition of a subject while using a central processing unit based electronic device comprising an input device that is capable of detecting an input from the subject's hand or finger. Such a method generally includes passively collecting an input device usage characteristics of a subject; processing the usage characteristic of the subject to provide a current usage characteristic result of the subject; and comparing the current usage characteristic result with a reference input device usage characteristic to determine whether there is any change in a clinical condition of the subject.

Typically, the input device is a pointing device. The input device usage characteristic can include: number of changes in direction on the x axis (x-flips); number of changes in direction on the y axis (y-flips); changes in speed; changes in precision; changes in distance; changes in idle time; changes in reaction time; changes in response time; changes in area under the curve; changes in maximum deviation; changes in additional distance; changes in normalized jerk; changes in keystroke transition time; changes in keystroke dwell time; changes in acceleration; changes in click latency; or a combination thereof.

In some embodiments, the reference usage characteristic comprises an average of a plurality of input device usage characteristics of the subject that are collected over a period of at least one hour. In other embodiments, the reference usage characteristic comprises an input device usage characteristic of the subject that is collected within 1 hour prior to the current usage characteristic result.

Yet another aspect of the invention provides a method for detecting a neuromotor disorder in a subject. The method comprises (a) determining an input device usage characteristic of a subject; and (b) analyzing the input device usage characteristic of the subject to a reference input device usage characteristic to determine whether the subject has a neuromotor disorder.

Often said neuromotor disorder is caused by a clinical condition comprising diabetes or a neurological disorder. In some particular embodiments, said neuromotor disorder is caused by hypoglycemia, a neurological disorder, a reaction to mediation, alcohol abuse or withdrawal, mercury poisoning, overactive thyroid, a clinical trial side-effect, peripheral neuropathy, early Parkinson's Disease, early Alzheimer's Disease, or liver failure.

In other embodiments, said reference input device usage characteristic comprises an input device usage characteristic of the subject collected within 1 hour prior to the input device usage characteristic of said step (a). Still in other embodiments, said reference input device usage characteristic comprises an input device usage characteristic of the subject determined within 24 hours prior to the input device usage characteristic of said step (a). Yet in other embodiments, said reference input device usage characteristic comprises an input device usage characteristic of the subject determined within seven days prior to the input device usage characteristic of said step (a). Still yet in other embodiments, said reference input device usage characteristic comprises an input device usage characteristic of the subject determined within a month prior to the input device usage characteristic of said step (a). Still yet in other embodiments, said reference input device usage characteristic comprises an input device usage characteristic of the subject determined within a year, typically within a month, often within a week, and still often within a few (e.g., less than 3) days prior to the input device usage characteristic of said step (a).

### BRIEF DESCRIPTION OF THE DRAWINGS

FIG. 1 is a graphic illustration of combined movement resulting when multiple answers catch the subject's attention.

FIG. 2 is an illustrative example of monitoring system question.

FIG. 3 is an illustration of monitoring system response analysis framework.

FIG. 4 is an example of a monitoring system test.

### DETAILED DESCRIPTION OF THE INVENTION

In a neuromotor disorder, the brain loses the ability to coordinate and control fine motor skills (eyes, fingers, hands, etc.). Tremors make fine motor skills more difficult to perform. The present inventors have found that even minute tremors can be precisely measured during a subject's use of a mouse or touch screen device or any other input devices.

At an even earlier stage (a pre-tremor stage), change in the subject's clinical conditions can be measured by detecting decreased motor performance in the subject. Thus, change in velocity, number of direction changes, distance, area under the curve, maximum deviation, and movement time while moving the mouse or finger or stylus on a touchscreen during a given task can indicate an early onset of a neuromotor disorder in the subject. Measuring a pointing device (e.g., mouse, touch screen, touch pad, joystick, trackball, stylus, etc.) performance at millisecond levels and pixel-level fidelity allows for subject's current performance to be compared to previously developed baselines to identify anomalies or change in subject's clinical condition (e.g., hand tremors). Because changes in motor performance (e.g., hand tremors) can be a result of a number of factors, in some embodiments the monitoring system of the invention simply indicates that a decrease in motor skill performance has occurred.

In other embodiments, the system of the invention is configured to refer the subject to a doctor for further testing. Table 1 summarizes some of the representative relevant features of input device usage characteristics that can indicate decreased motor performance that indicates early stages of onset of a medical condition or change in a clinical condition.

TABLE 1
Example mousing/touch screen indicators of
early neuromotor disorder

Number of changes in direction on the x axis (x-flips)
Number of changes in direction on the y axis (y-flips)
Changes in speed
Changes in precision
Changes in distance
Changes in idle time
Changes in reaction time
Changes in reaction time
Changes in applied pressure (i.e., touch interface)
Changes in area under the curve
Changes in maximum deviation
Changes in normalized jerk
Changes in additional distance
Changes in acceleration
Changes in transition time between keystrokes
Changes in dwell time of keystrokes
Changes in click latency

In some aspects of the invention, the system is based on the discovery by the present inventors that computer mouse, touch screen, keyboard, and other input devices measure motor movements with very fine detail and precision. In particular, the present inventors have discovered that an input device of a CPU based electronic device can be used to detect early-stages of medical or clinical conditions or a neuromotor disorder that affect motor skills that otherwise may be difficult, expensive, or invasive to identify. When conditions are detected in early stages, precautions and/or interventions can be made to improve the physical, emotional and/or financial impacts of the condition.

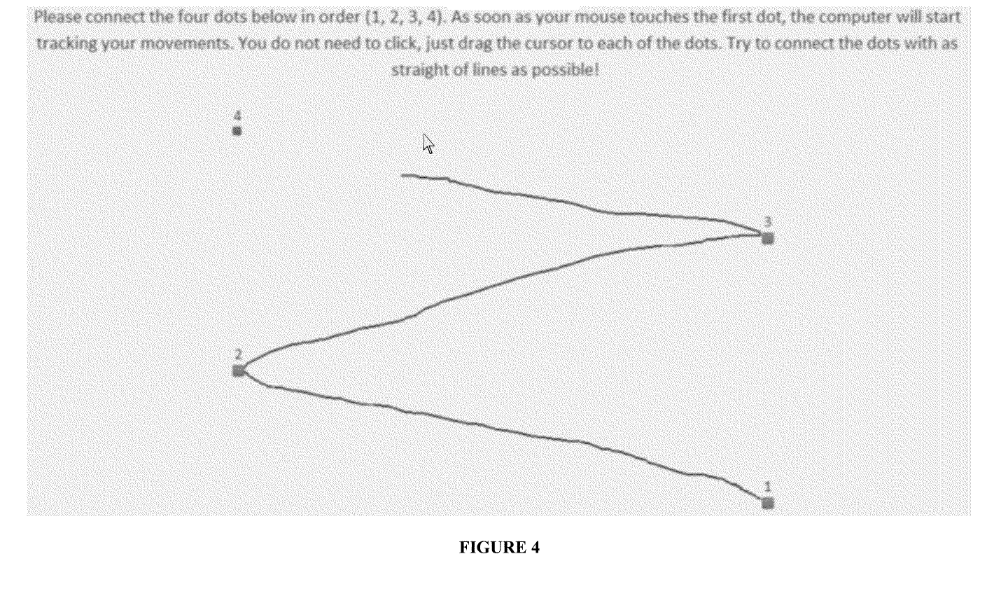

In some embodiments, the input device is a device that is based on a motor movement of the subject, typically one that requires a fine motor movement of the subject. In these embodiments, often the input device is a pointing device such as, but not limited to, mouse, touch screen, touch pad, trackball, joystick, laser pointer, stylus, or other input devices that require movement of the subject or the subject's hand or finger from point A to point B (see FIG. 4).

One particular aspect of the invention provides a system and/or a method of using the input device usage characteristic of the subject to detect or determine change in a medical or clinical condition of a subject. In some embodiments, the monitoring system utilizes at least two-prong solution that monitors hand-movements via an input device of a central processing unit (CPU) based-electronic device that has an input device based on the subject's motion (e.g., motion of hand and/or finger). The monitoring system of the invention provides early detection of medical or clinical condition or any changes thereof of the subject who is using the input device.

In some embodiments, the monitoring system identifies anomalies based on physical impediments—e.g., fine hand tremors or decreased performance due to a medical or a clinical condition. Exemplary medical or clinical conditions or a neuromotor disorder that can effect motor skill performance of a subject include, but are not limited to, hypoglycemia, a neurological disorder, reactions to mediations, alcohol abuse or withdrawal, mercury poisoning, overactive thyroid, a clinical trial side-effect, peripheral neuropathy, early Parkinson's Disease, early Alzheimer's Disease, and liver failure.

Yet in other embodiments, when tremors or decreased motor performance is determined, the system is configured to notify the subject, medical personnel, and/or other desired person (e.g., a family member) to investigate or determine the cause of the decreased motor performance.

Other aspects of the invention provide a medical or clinical condition surveying tool that establishes the credibility of responses through monitoring motor movements via an input device. Surveys or questionnaires are commonly used to assess a patient's medical state. For example, patients may be asked to complete a questionnaire regarding their health practices, adherence to medications, results of clinical trials, or outpatient condition. The monitoring system of the invention enhances these surveys by analyzing the credibility of patients' responses through the monitoring of motor movements via a computer input device (or smartphone, tablets, or any other CPU-based electronic device having an input device that is based on the motor movement of a user). The monitoring system of the invention enables earlier, more cost-effective, and less-obtrusive detection of possible medical or clinical conditions, and thereby can help mitigate the condition before further complications arise.

System Implementation: Different embodiments of the monitoring system can be implemented on the subject's electronic devices to detect early stages of a health condition. For example, a small rootkit is implemented on the subject's computer that continuously monitors mouse and keystroke behavior trends over time. The program can be configured to analyze the data locally or it can be configured to analyze the data remotely. If a decrease in motor performance is determined, an alert is generated (e.g., a pop-up window, text message back to the person, an automated phone call, a phone call from health care provider, email, etc.). In some embodiments, summary statistics is encrypted and sent to a remote server for archiving and data analysis to help improve health care in the future (e.g., help identify most effective interventions strategies for particular types of patients). In other embodiments, summary statistics is optionally encrypted and stored and analyzed within the subject's own computer.

In addition to or alternatively to the computer-based version of the monitoring system, in some embodiments, a smartphone-based application of the monitoring system is implemented. The smart phone app can alert the subject on a scheduled basis to perform a simple task using the touch screen (e.g., swiping their finger from one point to another, hitting a sequence of buttons, etc.). The movement is then analyzed to detect decreased or change in motor performance and thereby detect potentially early stages of any change in a medical or clinical condition of the subject.

The monitoring system of the invention has several benefits including, but not limited to, (i) detecting decreases in motor performance that may be indicative of an early-stage health condition; (ii) unobtrusive and continuous motor movement monitoring; (iii) conducting analysis using a smart phone that can be schedule to take place on regular intervals or at times when one is more susceptible to the medical condition (e.g., when a diabetic takes insulin, and thereby may experience hypoglycemia); (iv) evaluating the effects of clinical trials on motor movements; (v) evaluating the side-effects of medications; and (vi) |option of automatically alerting health care providers to provide assistance to subject, and analyze the data to discover more effective intervention techniques.

Survey Credibility Tool: In some aspects of the invention, the monitoring system can be used to verify the credibility of subject answers to questions (e.g., questions about adherence to prescriptions, clinical trial, etc.). This monitoring system can be a web-based or a stand-alone survey tool that elicits information to sensitive questions that relate to one's medical condition, and reliably detects whether one is being deceptive, concealing information, unsure how to answer, or experiencing a heightened emotional response to the question. Prior research on deception has established that being misleading or concealing information can be detected as observable behavioral changes when responding to questions regarding such events. Similar to the way a polygraph (lie detector) detects physiological changes in the body based on uncontrolled responses when answering sensitive questions, the present inventors have found that such responses can be detected through monitoring an input device (e.g., mouse and/or keystroke) usage characteristic (or behavior) when the subject is concealing or misrepresenting information. When such an anomaly is detected, the system can ask a follow-up question, or a healthcare personnel can be alerted to ask additional questions in this area.

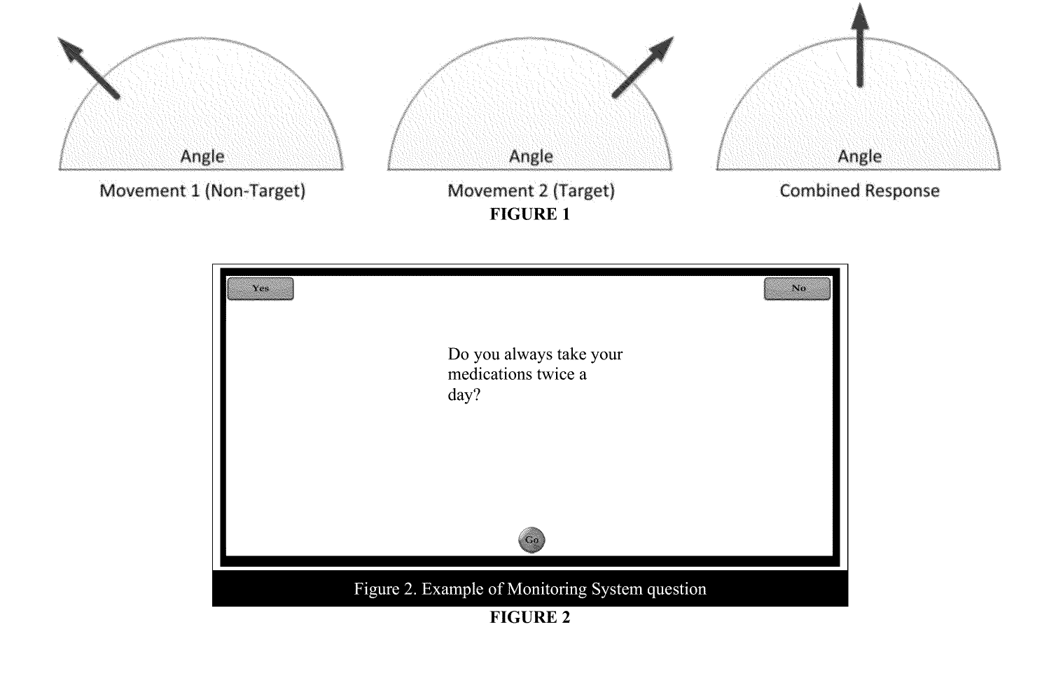

The present inventors have also discovered that input device usage characteristics (e.g., mouse and keyboard features) are useful in determining likelihood of deception (being misleading, fakery, etc.) to sensitive questions (i.e., questions about the health condition) administered on a computer. For example, the present inventors have found that when people see two or more answers to a question that catch their attention (e.g., a truthful answer and a deceptive answer that the person will ultimately choose), the mind automatically starts to program motor movements toward both answers simultaneously. To eliminate one of the motor movements (e.g., eliminate the movement towards admitting to an unhealthy practice), the mind begins an inhibition process so that the target movement can emerge. Inhibition is not immediate, however, but rather occurs over a short period of time depending on the degree both answers catch the respondents attention (up to ˜750 milliseconds or more). If movement begins before inhibition is complete, the movement trajectory is a product of motor programming to both answers. See FIG. 1. Thus, in a health question survey, when people are asked a health question that they do not want to answer truthfully, their mouse trajectory will be biased toward the truthful answer (measured on an x, y axis) on its way toward the deceptive answer. For innocent people, the opposite answer should not catch their attention to the same degree, and thus inhibition will occur more quickly and their mouse movements will be less biased.

In addition, being deceptive normally causes an increase in arousal and stress. Such arousal and stress causes neuromotor noise that interferes with people's fine motor skills (e.g., using the hand and fingers to move a mouse or use a touch screen to answer a question). As a result, the precision of mouse movements decreases when people are being deceptive, ceteris paribus. To reach the intended target (e.g., a deceptive answer in the Handshake survey), people automatically and subconsciously compensate for this decrease in precision through reducing speed and creating more adjustments to their movement trajectories based on continuous perceptual input. Thus, in health question surveys, the present inventors have found that people exhibit slower velocity, more adjustments (x and y flips), greater distance, and more hesitancy when being deceptive compared to when telling the truth.

In another example, the present inventors have found that people who are anticipating a certain question that they are planning on being deceptive on display different mouse movements on non-relevant questions compare to people who are not planning on being deceptive. In anticipation of a question that might reveal them, patients show a task-induced search bias: before answering a question, they take a fraction of a second longer to evaluate the question. After seeing that the question is not relevant, they then move more quickly to the truthful answer than people who are not a voiding a question. Table 2 summarizes examples of mousing features that distinguish a deceptive response.

TABLE 2
Examples of features that distinguish a deceptive response
(a subset of all features monitored)

| Statistic | Description |
|---|---|
| X | The X coordinates for each movement |
| Y | The Y coordinates for each movement |
| Z | The Z coordinate for each movement |
| Pressure | The pressure for each movement |
| Rescaled X | The X coordinates for the interaction normalized for screen |
| | resolution |
| Rescaled Y | The Y coordinates for the interaction normalized for screen |
| | resolution |
| X Average | The X coordinates averaged in buckets of 75 ms |
| Y Average | The Y coordinates averaged in buckets of 75 ms |
| X Norm | The X coordinates time normalized |
| Y Norm | The Y coordinates time normalized |
| Pressure | The pressure applied to the mouse for every raw recording |
| Timestamps | The timestamp for every raw recording |
| Click Direction | Whether the mouse button was pushed down (d) or |
| | released (u) for every time an action occurred with the |
| | mouse button |
| Click X | The X coordinates for each mouse click event |
| Click Y | The Y coordinates for each mouse click event |
| Click Rescaled X | The X coordinates for each mouse click event normalized |
| | for screen resolution |
| Click Rescaled Y | The Y coordinates for each mouse click event normalized |
| | for screen resolution |
| Click Pressure | The pressure applied to the mouse for every raw recording |
| Click timestamps | The timestamp for every mouse click event |
| Acceleration | The average acceleration for each 75 ms |
| Angle | The average angle for each 75 ms |
| Area Under the Curve (AUC) | The geometric area between the actual mouse trajectory |
| | and the idealized response trajectory (i.e., straight lines |
| | between users' mouse clicks); it is a measure of total |
| | deviation from the idealized trajectory. |
| Additional AUC | The AUC minimum the minimum AUC |
| Overall Distance | The total distance traveled by the mouse trajectory |
| Additional Distance | The distance a users' mouse cursor traveled on the screen |
| | minus the distance that it would have required to traveling |
| | along the idealized response trajectory (i.e. straight lines |
| | between users' mouse clicks), |
| Distance Buckets | Distance traveled for each 75 ms |
| X Flips | The number of reversals on the x axis |
| Y Flips | The number of reversals on the y axis |
| Maximum Deviation | The largest perpendicular deviation between the actual |
| | trajectory and its idealized response trajectory (i.e., straight |
| | lines between users' mouse clicks), |
| Speed Buckets | Average speed for each 75 ms |
| Overall Speed | Average overall speed |
| Idle Time | if there is a change in time greater than 200 ms but no |
| | movement, this is counted as idle time |
| Idle Time on Same Location | If there is a change in time but not a change in location, |
| | this mean an event other than movement triggered a |
| | recording (e.g., such as leaving the page, and other things). |
| | The time in this event is summed. |
| Idle Time On 100 Distance | If there is a change in distance greater than 100 between |
| | two points, this may indicate that someone left the screen |
| | and came back in another area |
| Total Time | Total response time |
| Click Mean Speed | The mean speed of users click |
| Click Median Speed | The median speed of users click |
| Click Mean Latency | The mean time between when a user clicks down and |
| | releases the click |
| Click Median Latency | The median time between when a user clicks down and |
| | releases the click |
| Answer Changes | The number of times an answer was selected; if over 1, the |
| | person changed answers |
| Hover Changes | The number of times an answer was hovered; if over 1, the |
| | person hovered over answers they didn't chose |
| Hover Region | The amount of time a person overs over a region |
| Return Sum | The number of times a person returns to a region after |
| | leaving it |
| Dwell | The measurement of how long a key is held down |
| Transition | Time between key presses |
| Rollover | The time between when one key is released and the |
| | subsequent key is pushed |

System Implementation: In some embodiments, the monitoring system of the invention asks health-related question on a computer and requires respondents to drag a button on the bottom of the screen to ‘yes’ or ‘no’ to answer. Responses are analyzed with both with-in subject comparisons as well as by comparing responses with the aggregate responses of other subjects. For instance, FIG. 2 is an example of a medical adherence question—“Do you always take your medications twice a day?” In this example, the subject must move the mouse from the lower middle of the screen to the “No” answer to deny taking the medication twice a day or to “Yes” to confirm taking the medication. Mouse movements are captured while the subject is answering the question and compared to the subject's baseline (how the subject moves the mouse on truthful responses) and to a control group baseline (how other people normally move the mouse on this question) to detect deception.

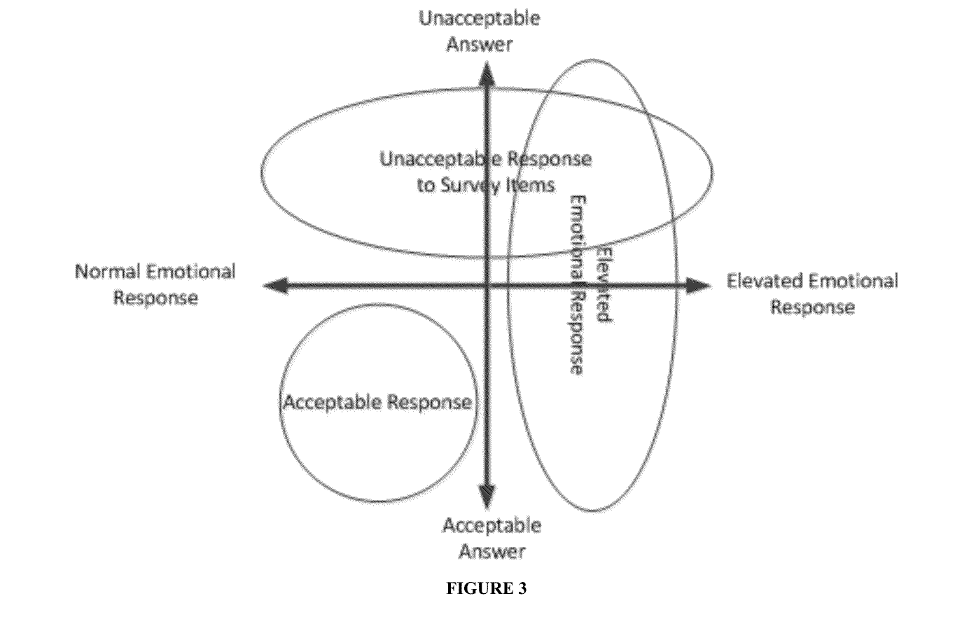

Survey items have acceptable and non-acceptable ranges of responses (see FIG. 3); acceptable response can be determined by the organization involved in the survey. For example, for the question “Do you always take your medications twice a day?”—“Yes” is the acceptable answer and “No” is an unacceptable answer. The monitoring system can also use other question formats (e.g., radio buttons, sliders, Likert scales, etc.). By observing both the answer provided by the subject and using the input device usage characteristics (e.g., the mouse and keyboard behavior) to detect changes in emotional response, one can make four potential observations about a response to a survey item. See, FIG. 3. Some aspects of determining how to obtain input device usage characteristics of the subject is disclosed in U.S. Provisional Patent Application No. 61/837,153, filed Jun. 19, 2013, and U.S. Pat. No. 8,230,232, issued to Ahmed et al., which are incorporated herein by reference in their entirety. In FIG. 3, Lower Left Quadrant: Answer was within acceptable range with normal emotional response (i.e., no action is necessary); Upper Left Quadrant: Answer is outside acceptable range with normal emotional response Upper Right Quadrant: Answer is outside acceptable range with elevated emotional response; Lower Right Quadrant: Answer was within acceptable range, however, with an elevated emotional response (i.e., a deceptive answer; follow-up questioning should be performed).

Several functionalities can be implemented in the system of the invention to facilitate accurate and reliable analysis of mouse movements. For example, (i) Data is time-normalized (e.g., all trajectories are evenly split into 101 equal buckets) to compare trajectories between respondents for detecting deception; (ii) Data is averaged into 75 ms duration intervals to account for differences in computers speeds and mouse characteristics within subjects; (iii) Data is rescaled to a standard scale so we can the trajectories of respondents who used different screen resolutions (iv) Respondents are required to start moving their mouse or finger before an answer is shown, so that we can capture a respondent's initial movements as soon as they see the answer; (v) If respondents stop moving their mouse or finger or stop dragging an answer, an error is shown; (vi) To help respondents get use to the testing format and improve the performance of the evaluation, a tutorial and practice test are provided; (vii) All items (sensitive and control items) are pilot tested to make sure the average person respond as intended (viii) A tree-like questioning framework is implemented to ask follow-up questions when deception is detected; (ix) All mousing data is sent to a server data server via a web service to be analyzed and determine deception. This reduces the likelihood that data can be tampered with during the analysis; (x) A secure management dashboard is implemented to visualize the results; (xi) Probabilities of deception are calculated based on multi-tiered testing; (xii) Different features of deception are extracted for different devises (desktop, iPad, etc.).

In some embodiments of the invention, the monitoring system introduces a lightweight and easily deployable system for quickly identifying deception in health questionnaires. Other advantages of the monitoring system of the invention include, but are not limited to, (i) Easy and inexpensive to deploy to a large number of people simultaneously; (ii) A data capture process runs in the background during survey administration, while analysis takes place on a separate and secure remote system; (iii) Behavioral sensing data (e.g., keyboard and mouse usage) is gathered in an unobtrusive manner with no adverse effect to the user; (iv) Users need not be aware of the data collection that is taking place; (v) Unlike systems that rely on linguistic features, the system's behavioral analysis approach is language agnostic (e.g., the detection methodology will work with English, Spanish, Arabic, etc.) because it relies on system usage patterns rather than message content; (vi) The system is not easily fooled, as heightened emotions that would trigger anomalous event typically manifests itself as subtle differences in typing or mouse movement behavior that occurs between 20 and 100 milliseconds. Attempts to modify one's keystroke or mouse use would be flagged as abnormal, thus identifying individuals attempting to fool the system; (vii) The system is not subject to biases that are common in face-to-face investigations.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

In 2011, 25.8 million children and adults in the United States—8.3% of the population—had diabetes. Complications of diabetes include heart disease and stroke, high blood pressure, blindness, kidney disease, neuropathy, and amputations, contributing to a total of 231,404 deaths in United States during 2011 (2011 National Diabetes Fact Sheet). The cost of diabetes in the United States in 2012 was $245 billion (“Economic Costs of Diabetes in the U.S. in 2012”). However, improvement in care in recent decades has enabled people with diabetes to live long, healthy lives. Careful adherence to medical prescriptions (e.g., insulin injections, eating recommendations), however, is essential for a healthy life. For example, diabetics who were least compliant with their directed medication usage were twice as likely to be hospitalized compared to those who were most compliant, and their total health-care costs were nearly double (Epstein, 2005).

One common complication of diabetic treatments (e.g., insulin injections) is hypoglycemia—i.e., low blood sugar levels (The DCCT Research Group 1997). Hypoglycemia is extremely common; it is believed that approximately 90% of all patients who receive insulin have experienced hypoglycemic episodes. Surveys investigating the prevalence of hypoglycemia have found that intensively treated Type 1 diabetic patients are three times more likely to experience severe hypoglycemia and coma than conventionally treated patients; experiencing up to 10 episodes of symptomatic hypoglycemia per week, and severe temporarily disabling hypoglycemia at least once a year. Two to four percent of deaths of people with Type 1 diabetes is believed to be caused by hypoglycemia, and 70-80% of patients with Type 2 diabetics using insulin experience hypoglycemia.

To help detect and thereby intervene when hypoglycemia events are occurring, a monitoring system that resides on diabetics' computers, smart phones, and other electronic devices is used. The monitoring system of the invention monitors how the subject moves the subject's hand and/or fingers (e.g., how the subject moves the mouse, how the subject uses a finger on a touch screen, etc.) and thereby indicates when the subject may have low blood glucose levels. Hand tremors are a common early symptom of hypoglycemia (typically when blood sugar drops below 60 to 70 mg/dl). Computer mice and touch screens measure movements with very fine detail and precision, and thus have potential to detect hypoglycemia in its early stages. When detected, interventions is initiated (e.g., warnings, text-messages, automated phone calls from a health care provider, etc.) to proactively intervene during the early stages of an episode, before more life-threatening complications occur that require more intrusive interventions such as emergency care and hospitalization. Also, the monitoring system database can be mined to find common characteristics of subjects that don't manage their situation well, helping to identify other possible intervention strategies.

Hand Tremors and Blood Sugar: Hand tremors are an early symptom of hypoglycemia. When one has low blood glucose, the brain loses the ability to coordinate and control fine motor skills (eyes, fingers, hands, etc.). When the tremor is present, fine motor skills become more difficult to perform. From a measurement perspective, minute tremors are precisely measured using a mouse or touch screen device by recording direction changes on the x and y axis.

At an even earlier stage (a pre-tremor stage), the monitoring system of the invention is used to determine hypoglycemia by detecting decreased mouse or touchpad performance. Directed hand movements (e.g., moving the mouse cursor to a target) are typically made up of an initial movement, followed by one or more secondary movements to reach a target. When hand movements become more difficult to perform because of physical or mental impediments, the body automatically adjusts the movement to compensate for the impediment. First, the body decreases the velocity of the movement. Decreasing the velocity of the movement increases its accuracy in reaching a destination. Second, the body increases the number of secondary sub-movements, or makes more adjustments to the movement trajectory based on continuous visual input (which increases direction changes and overall distance). The mind will optimize the velocity and number of submovements to reach the destination in the least amount of time.

A change in velocity, number of direction changes, distance, and movement time while moving the mouse or finger on a touchscreen during a given task indicates the early onset of hypoglycemia in a diabetic. Measuring mouse and touch performance at millisecond levels and pixel-level fidelity allows for subject's current performance to be compared to previously developed baselines (i.e., the reference input device usage characteristics). Relevant features of mouse and touch screen usage that can indicate early stages of hypoglycemia is disclosed in Table 1.

In addition to detecting hypoglycemia, the monitoring system of the invention can help detect hand tremors related to other health or clinical conditions: neurological disorders, reactions to mediations (such as amphetamines, corticosteroids, and drugs used for certain psychiatric disorders), alcohol abuse or withdrawal, mercury poisoning, overactive thyroid, and liver failure.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

1. A monitoring system for detecting a change in a clinical condition of a subject while using a central processing unit based electronic device comprising an input device that is capable of detecting motion of a subject's hand or finger, said system comprising:
a data interception unit configured to intercept inputs from a user, wherein the data interception unit is configured to passively collect an input device usage characteristics;
a behavior analysis unit operatively coupled to said data interception unit to receive the passively collected input device usage characteristic, and
a behavior comparison unit operatively coupled to said behavior analysis unit,
wherein said monitoring system dynamically monitors and passively collects input device usage characteristic information, and translates said input device usage characteristic information into representative data, stores and compares different results, and outputs a result associated with changes in the clinical condition of the subject.

2. The monitoring system of claim 1, wherein the clinical condition comprises hypoglycemia, a neurological disorder, a reaction to mediation, alcohol abuse or withdrawal, mercury poisoning, overactive thyroid, a clinical trial side-effect, peripheral neuropathy, early Parkinson's Disease, early Alzheimer's Disease, or liver failure.

3. The monitoring system of claim 2, wherein the clinical condition is hypoglycemia.

4. The monitoring system of claim 1, wherein the input device is a pointing device.

5. The monitoring system of claim 4, wherein the pointing device comprises mouse, touch screen, track ball, touch pad, joystick, stylus, or a combination thereof.

6. The monitoring system of claim 4, wherein said pointing device usage characteristic comprises: number of changes in direction on the x axis (x-flips); number of changes in direction on the y axis (y-flips); changes in speed; changes in precision; changes in distance; changes in idle time; changes in reaction time; changes in response time; changes in area under the curve; changes in maximum deviation; changes in additional distance; changes in normalized jerk; changes in keystroke transition time; changes in keystroke dwell time; changes in acceleration; changes in click latency; or a combination thereof.

7. The monitoring system of claim 1, wherein said behavior comparison unit is further configured to alert the subject when the result indicates hypoglycemic condition or other condition that influence motor control.

8. The monitoring system of claim 7, wherein said alert comprises a pop-up window, text message to the subject, an automated phone call, a phone call from health care provider, email, or a combination thereof.

9. The monitoring system of claim 1, wherein said monitoring system is configured in a computer, a smartphone, or any other central processing unit based electronic device comprising an input device that is capable of detecting a motion input from the subject's hand or finger.

10. The monitoring system of claim 1, wherein said monitoring system is suitably configured for real-time monitoring.

11. A method of determining change in a clinical condition of a subject while using a central processing unit based electronic device comprising an input device that is capable of detecting an input from the subject's hand or finger, said method comprising:
passively collecting an input device usage characteristics of a subject;
processing the usage characteristic of the subject to provide a current usage characteristic result of the subject; and
comparing the current usage characteristic result with a reference usage characteristic to determine whether there is any change in a clinical condition of the subject.

12. The method of claim 11, wherein said input device is a pointing device.

13. The method of claim 12, wherein said input device usage characteristic comprises: number of changes in direction on the x axis (x-flips); number of changes in direction on the y axis (y-flips); changes in speed; changes in precision; changes in distance; changes in idle time; changes in reaction time; changes in response time; changes in area under the curve; changes in maximum deviation; changes in additional distance; changes in normalized jerk; changes in keystroke transition time; changes in keystroke dwell time; changes in acceleration; changes in click latency; or a combination thereof.

14. The method of claim 11, wherein the reference usage characteristic comprises an average of a plurality of input device usage characteristics of the subject that are collected over a period of at least one hour.

15. The method of claim 11, wherein the reference usage characteristic comprises an input device usage characteristic of the subject that is collected within 1 hour prior to the current usage characteristic result.

16. A method for detecting a neuromotor disorder in a subject, said method comprising:
(a) determining an input device usage characteristic of a subject; and
(b) analyzing the input device usage characteristic of the subject to a reference input device usage characteristic to determine whether the subject has a neuromotor disorder.

17. The method of claim 16, wherein said neuromotor disorder is caused by a clinical condition comprising diabetes or a neurological disorder.

18. The method of claim 16, wherein said neuromotor disorder is caused by hypoglycemia, a neurological disorder, a reaction to mediation, alcohol abuse or withdrawal, mercury poisoning, overactive thyroid, a clinical trial side-effect, peripheral neuropathy, early Parkinson's Disease, early Alzheimer's Disease, or liver failure.

19. The method of claim 16, wherein said reference input device usage characteristic comprises an input device usage characteristic of the subject collected within 1 hour prior to the input device usage characteristic of said step (a).

20. The method of claim 16, wherein said reference input device usage characteristic comprises an input device usage characteristic of the subject determined within 24 hours prior to the input device usage characteristic of said step (a).

21-23. (canceled)
